(19) United States
(12) Patent Application Publication
(10) Pub. No.: US 2012/0283624 A1
(43) Pub. Date: Nov. 8, 2012

(54) Drug Eluting Device and Method of Use Thereof

(76) Inventor: Gary Bradford Shirley, Bloomington, IN (US)

(21) Appl. No.: 13/463,023

(22) Filed: May 3, 2012

Related U.S. Application Data
(60) Provisional application No. 61/482,765, filed on May 5, 2011.

(51) Int. Cl. A61M 37/00; A61L 29/08; A61L 29/16

(52) U.S. Cl. 604/22

(57) ABSTRACT

One aspect provides a balloon catheter including a cutting element having a reservoir. In one embodiment, the reservoir includes a bioactive. Another aspect provides a method for using the balloon catheter for treating a patient by breaking hardened plague on the vessel wall and dilating the vessel. In one embodiment, the bioactive is delivered to the vessel wall.

### RELATED APPLICATIONS

The present patent application claims the benefit of U.S. Provisional Patent Application No. 61/482,765, filed May 5, 2011, the contents of which are hereby incorporated by reference.

### FIELD OF THE INVENTION

The present invention relates generally to medical devices including a bioactive and more particularly to balloon catheters and their use to dilate narrowed portions of a vessel having a hardened lesion.

### BACKGROUND

Balloon catheters are widely used in the medical profession for various intraluminal procedures. One common procedure involving the use of a balloon catheter relates to angioplasty dilation of coronary or other arteries suffering from stenosis (i.e., a narrowing of the arterial lumen that restricts blood flow).

Although balloon catheters are used in many other procedures as well, coronary angioplasty using a balloon catheter has drawn particular attention from the medical community because of the growing number of people suffering from heart problems associated with stenosis. This has lead to an increased demand for medical procedures to treat such problems. Angioplasty procedures have become a popular alternative for treating coronary stenosis because angioplasty procedures are considerably less invasive than other alternatives. For example, stenosis of the coronary arteries has traditionally been treated with bypass surgery. In general, bypass surgery involves splitting the chest bone to open the chest cavity and grafting a replacement vessel onto the heart to bypass the blocked, or stenosed, artery. However, coronary bypass surgery is a very invasive procedure that is risky and requires a long recovery time for the patient.

To address the increased need for coronary artery treatments, the medical community has turned to angioplasty procedures, in combination with stenting procedures, to avoid the problems associated with traditional bypass surgery. Typically, angioplasty procedures are performed using a balloon-tipped catheter that may or may not have a stent mounted on the balloon (also referred to as a stented catheter). The physician performs the angioplasty procedure by introducing the balloon catheter into a peripheral artery (commonly one of the leg arteries) and threading the catheter to the narrowed part of the coronary artery to be treated. During this stage, the balloon is deflated and collapsed onto the shaft of the catheter in order to present a low profile which may be passed through the arterial lumens.

Once the balloon is positioned at the narrowed part of the artery, the balloon is expanded by pumping a mixture of saline and contrast solution through the catheter to the balloon. As a result, the balloon presses against the inner wall of the artery to dilate it. If a stent is mounted on the balloon, the balloon inflation also serves to expand the stent and implant it within the artery. After the artery is dilated, the balloon is deflated so that it once again collapses onto the shaft of the catheter. The balloon-tipped catheter is then retracted from the arteries. If a stent is mounted on the balloon of the catheter, the stent is left permanently implanted in its expanded state at the desired location in the artery to provide a support structure that prevents the artery from collapsing back to its pre-dilated condition. On the other hand, if the balloon catheter is not adapted for delivery of a stent, either a balloon-expandable stent or a self-expandable stent may be implanted in the dilated region in a follow-up procedure.

Although the treatment of stenosed coronary arteries is one common example where balloon catheters have been used, many other uses are also possible. For example, balloon catheters can have application in the treatment of blockages of the peripheral blood vessels, esophagus, trachea, colon, biliary tract, urinary tract and at other locations in the body. Other applications include the treatment of carotid artery stenosis, the narrowing of the carotid arteries, which are the main arteries in the neck that supply blood to the brain. Carotid artery stenosis (also called carotid artery disease) is a relatively high risk factor for ischemic stroke. The narrowing is usually caused by plaque build-up in the carotid artery. Plaque forms when cholesterol, fat and other substances form in the inner lining of an artery. This formation is called atherosclerosis.

One problem that may be encountered with conventional angioplasty techniques is the proper dilation of stenosed regions that are hardened and/or have become calcified. Stenosed regions may become hardened for a variety of reasons, such as the buildup of atherosclerotic plaque or other substances. Hardened regions of stenosis can be difficult to completely dilate using conventional balloons because hardened regions tend to resist the expansion pressures applied by conventional balloon catheters.

Angioplasty cutting devices offer a method for treating hardened regions. Such devices include an angioplasty balloon having one of more cutting surfaces present on the balloon surface. Upon expansion of the balloon, the cutting surfaces are configured to contact the hardened vessel wall and to break the plague, allowing further expansion of the vessel. One such cutting device is disclosed in U.S. publication number 2006/0173487A1, published Aug. 3, 2006.

While angioplasty presently enjoys wide use, it suffers from two major problems. First, the blood vessel may suffer acute occlusion immediately after or within the initial hours after the dilation procedure. Such occlusion is referred to as “abrupt closure.” Abrupt closure occurs in perhaps five percent or so of the cases in which angioplasty is employed, and can result in myocardial infarction and death if blood flow is not restored promptly. The primary mechanisms of abrupt closures are believed to be elastic recoil, arterial dissection and/or thrombosis. It has been postulated that the delivery of an appropriate agent (such as an antithrombotic) directly into the arterial wall at the time of angioplasty could reduce the incidence of thrombotic acute closure, but the results of attempts to do so have been mixed.

A second major problem encountered in angioplasty is the re-narrowing of an artery after an initially successful angioplasty. This re-narrowing is referred to as “restenosis” and typically occurs within the first six months after angioplasty. Restenosis is believed to arise through the proliferation and migration of cellular components from the arterial wall, as well as through geometric changes in the arterial wall referred to as “remodeling.”

The delivery of appropriate bioactives directly into the arterial wall offers a route to interrupt the cellular and/or remodeling events leading to restenosis. Bioactive coated stent devices have been employed for this purpose. For example, stents coated with sirolimus, paclitaxel or similar bioactives have been employed for this purpose.

It would be also be desirable to develop non-stenting devices and methods for reliably delivering suitable bioactives directly into a body portion during or following balloon angioplasty, so as to treat or prevent such conditions and diseases, for example, to prevent abrupt closure and/or restenosis of a body portion such as a passage, lumen or blood vessel.

### SUMMARY

One aspect provides a balloon catheter having a cutting element including a reservoir. Another aspect provides a method for treating a patient by breaking hardened plague on a vessel wall and dilating the vessel. In one embodiment, a bioactive is delivered to the vessel wall from the reservoir.

In one embodiment, the balloon catheter includes a shaft and an inflatable balloon mounted at the distal end of the shaft. A lumen extends through the shaft and is in fluid communication with the interior of the balloon. A cutting element including a reservoir is positioned on the exterior surface of the balloon. In one embodiment, the balloon includes a wall of variable thickness, the cutting element forming a thickened portion of the wall. The reservoir is present as a depression in the thickened portion.

In various embodiments, a bioactive, such as a taxane, a taxane derivative, everolimus, zotarolimus, paclitaxel, rapamycin, a rapamycin derivative, an antisense oligonucleotide or a mTOR inhibitor, is present in the reservoir. In one embodiment, the bioactive is paclitaxel. The bioactive can also be coated on a portion of the external surface of the balloon other than the reservoir.

Another aspect provides a method using the balloon catheter for delivering a bioactive to a vessel wall. The method includes inserting and positioning the device within the vessel and inflating the balloon, whereby the cutting element breaks hardened plague on the vessel wall and expands the vessel. The cutting element can be maintained in contact with the vessel wall for a time period sufficient to deliver at least a portion of the bioactive to the vessel wall.

### BRIEF DESCRIPTION OF THE DRAWINGS

The invention may be more fully understood by reading the following description in conjunction with the drawings, in which:

FIGS. 1A and 1B are illustrations showing embodiments of a balloon having one or more cutting elements. In FIG. 1A, a cutting element forms a spiral on the external surface of the balloon. In FIG. 1B, cutting elements extend along the external surface of the balloon.

FIG. 2A is an illustration showing a transverse cross-sectional view of the balloon catheter of FIG. 1B cut through balloon 121. FIG. 2B is an illustration showing an enlarged cross-sectional view of a cutting element including a reservoir containing a bioactive.

FIGS. 3A, 3B and 3C are illustrations showing transverse cross-sectional views of various embodiments of a cutting element having a reservoir.

FIG. 4 is an illustration showing another embodiment of a balloon having cutting elements.

FIGS. 5A and 5B are expanded partial transverse cross-sectional views of an embodiment of a balloon catheter having a cutting element.

### DEFINITIONS

Unless otherwise defined, all technical and scientific terms used herein have the same meaning as commonly understood by one of ordinary skill in the art to which this invention pertains. In case of conflict, the present document, including definitions, will control. Preferred methods and materials are described below, although methods and materials similar or equivalent to those described herein can be used in the practice or testing of the present invention. The materials, methods, and examples disclosed herein are illustrative only and not intended to be limiting.

As used herein the terms “comprise(s),” “include(s),” “having,” “has,” “can,” “contain(s),” and variants thereof, are intended to be open-ended transitional phrases, terms, or words that do not preclude the possibility of additional acts or structures. The present invention also contemplates other embodiments “comprising,” “consisting of” and “consisting essentially of,” the embodiments or elements presented herein, whether explicitly set forth or not.

### DETAILED DESCRIPTION

For the purposes of promoting an understanding of the principles of the invention, reference will now be made to the embodiments illustrated in the drawings and specific language will be used to describe the same. It will nevertheless be understood that no limitation of the scope of the invention is thereby intended, and alterations and modifications in the illustrated device, and further applications of the principles of the invention as illustrated therein are herein contemplated as would normally occur to one skilled in the art to which the invention relates.

One aspect of the present invention provides a medical device including an expandable balloon having a least one cutting element on an external surface of the balloon. In one embodiment, the cutting element includes a reservoir. In certain embodiments, at least one bioactive is contained in the reservoir. As used herein, the term “bioactive” refers to any pharmaceutically active agent that produces an intended therapeutic effect on the body to treat or prevent conditions or diseases. Another aspect of the present invention provides a method for using the medical device for dilating hardened regions of a stenosis in a body vessel and for delivering the bioactive to the wall of the vessel.

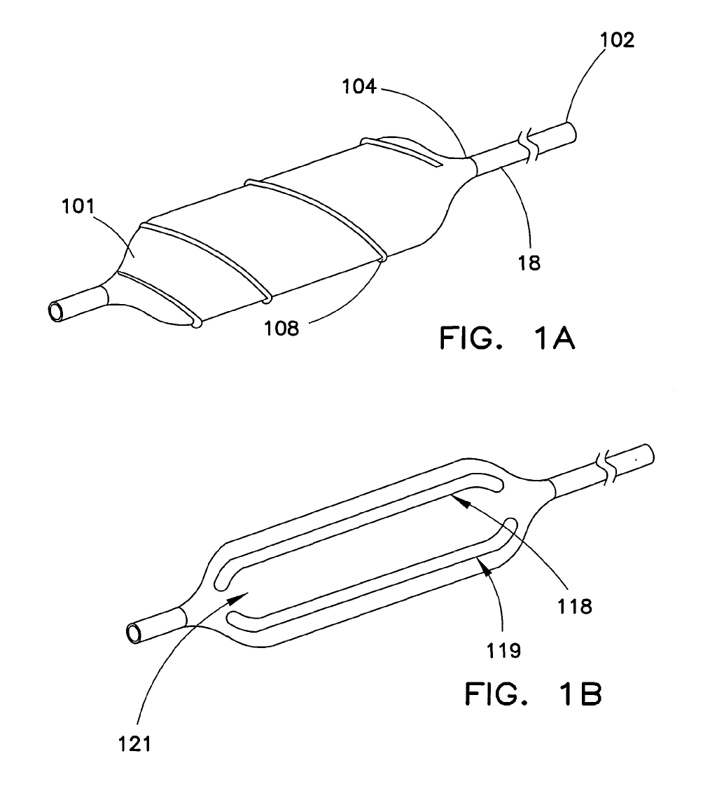

FIG. 1A illustrates one embodiment of a balloon catheter having a cutting element. The catheter includes a catheter shaft 18 extending from a proximal end 102 to balloon 101. A balloon inflation lumen (not shown) extends from the proximal end of catheter shaft 18 and is in fluid communication with the interior of balloon 101. Balloon 101, which is illustrated in an expanded state, is mounted on the distal end 104 of the shaft. In certain embodiments, catheter shaft 18 also includes a guidewire lumen. Spiral cutting element 108 extends around and along the surface of balloon 101, forming a ridge extending across the surface of the balloon.

FIG. 1B illustrates another embodiment of a balloon catheter of the present invention. In this embodiment, a number of cutting elements, of which cutting elements 118 and 119 are shown, overlay the outside surface of balloon 121 and extend from near the distal end of the balloon to near the proximal end of the balloon. In certain embodiments, the balloon catheter includes 1, 2, 3, 4, 5, 6, 7, 8, 9, 10 or more cutting elements.

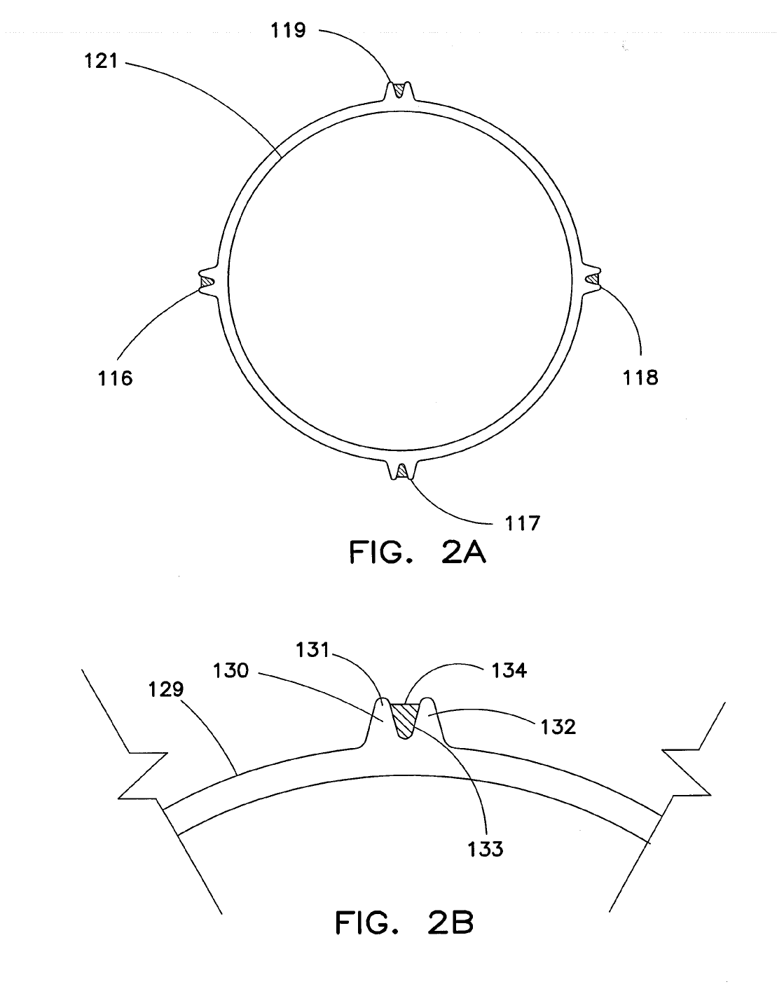

FIG. 2A illustrates a cross sectional view of balloon 121. Four cutting elements 116, 117, 118 and 119 are present on the surface of the balloon. In this embodiment, the four cutting elements are spaced evenly apart on the circumference of balloon 121. However, in other embodiments, more or fewer cutting elements can be present and the separation of the cutting elements need not be even. FIG. 2B illustrates an enlarged cross sectional view of one embodiment of a cutting element. In this embodiment, cutting element 130 may be a cutting element such as those illustrated in FIGS. 1A and 1B. Cutting element 130 includes cutting element walls 131 and 132 separated by reservoir 133 formed as a depression at the apex of the cutting element. In one embodiment, reservoir 133 contains a bioactive 134.

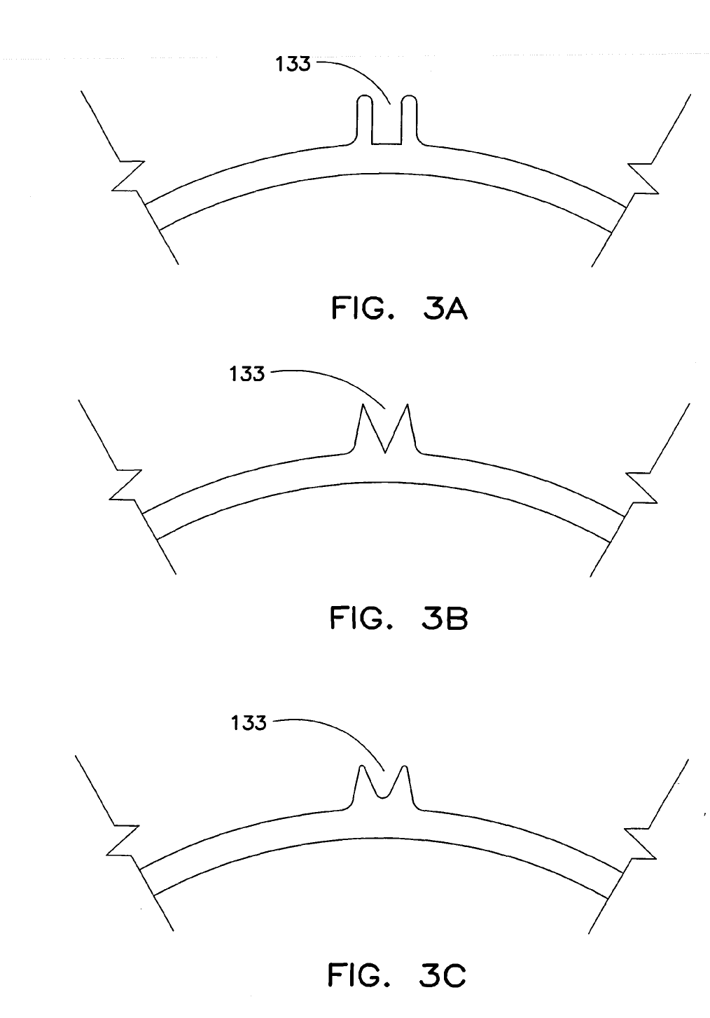

FIGS. 3A-3C illustrate various embodiments of the cross section of reservoir 133. In these embodiments, the bioactive is not shown. Reservoir may have a square bottom as illustrated in FIG. 3A. Alternatively, the reservoir may be “V” shaped or rounded as illustrated in FIG. 3B or 3C respectively. Of course, other reservoir shapes are encompassed within the scope of the present embodiments. The only requirement is that the reservoir is adequate to contain a bioactive.

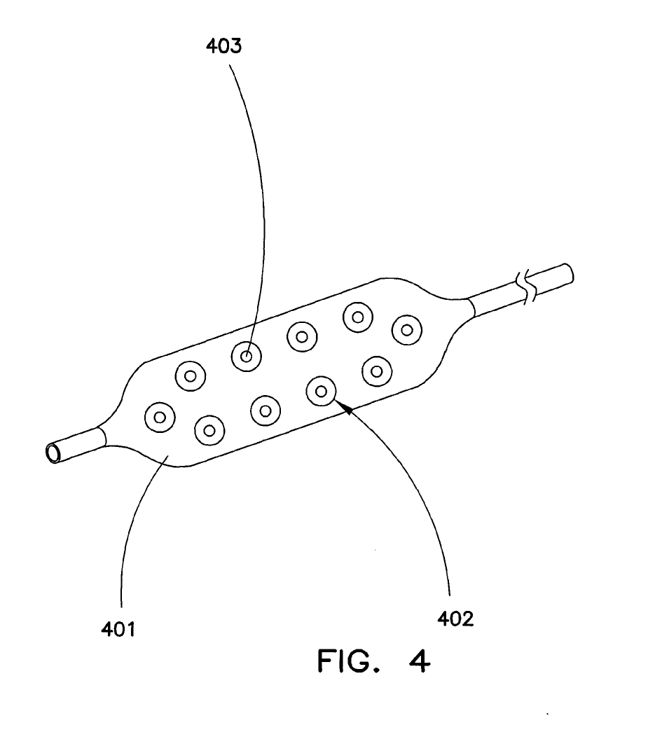

FIG. 4 illustrates another embodiment of a balloon catheter of the present invention. In this embodiment, a plurality of cutting elements 402 having reservoirs 403 are positioned on the outside surface of balloon 401. In this embodiment, the cutting elements are shown as being approximately circular and including an approximately circular wall surrounding a reservoir. However, the present embodiments encompass cutting elements and reservoirs of other shapes including, but not limited to, oval, irregular shaped, square, triangular and rectangular. In certain embodiments, cutting elements 402 are positioned in a regular pattern on the surface of balloon 401. In other embodiments, cutting elements are present only on certain portions of the surface, while other portions of the surface are free on cutting elements.

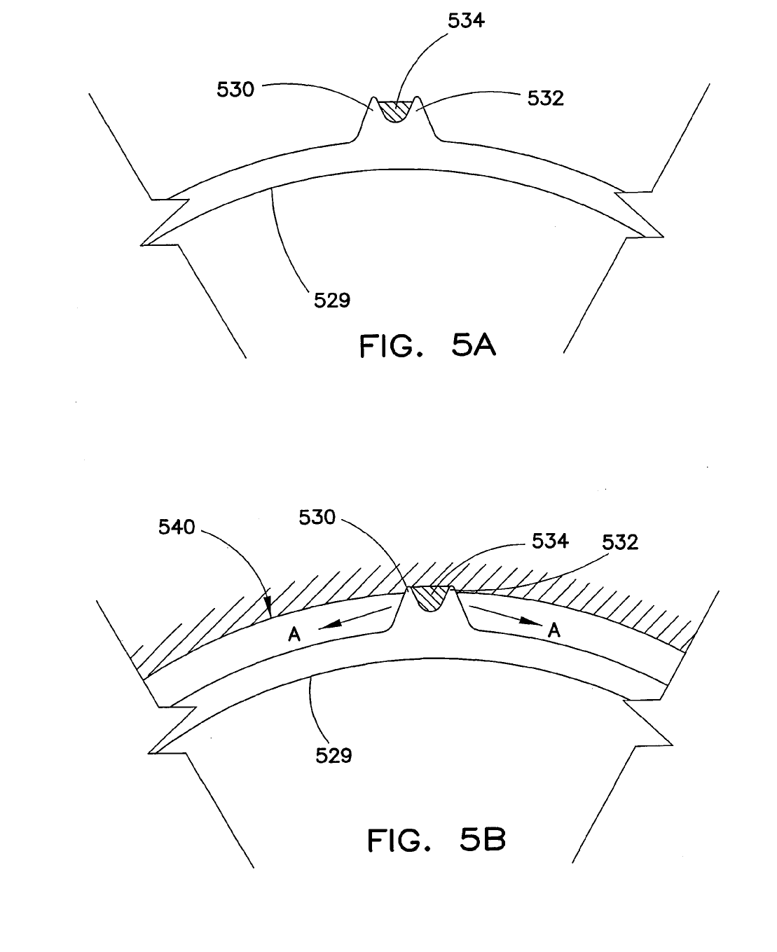

In another embodiment, shown in FIGS. 5A and 5B, walls 530 and 532 of a cutting element on the surface of balloon 529 are shaped to move apart when balloon 529 is inflated to near its maximum expansion. FIG. 5B illustrates balloon 529 in the expanded configuration. Here walls 530 and 532 have moved in the direction of arrows “A” as balloon 529 expanded to its inflated configuration to contact vessel wall 540. In one embodiment, this movement assists in the delivery of bioactive 534 from the reservoir to vessel wall 540.

In one embodiment, the balloon is manufactured from a silicone. Other biocompatible materials can also be used. Such materials include, but are not limited to, biocompatible polymers such as polyethyleneterepthalate (PET), polyvinyl chloride, polypropylene, polyethylene, polyurethanes, nylons, polyesters, latex, natural rubber, synthetic rubber, elastomers and mixtures or copolymers of these materials.

In certain embodiments, the cutting element(s) are formed from the same material as the remainder of the balloon. For example, the cutting elements can be formed during the balloon molding process, i.e. the cutting elements and the balloon are molded as an integral unit. In these embodiments, the balloon wall is of variable thickness, the walls of the cutting elements being thicken portions of the wall.

In certain embodiments, a bioactive is present within the reservoir of the cutting element. However, the present embodiments also encompass devices, and methods of using devices, in which the reservoir does not contain a bioactive. In other embodiments, the bioactive is contained within the reservoirs of the cutting elements are well as being present on other portions of the surface of the balloon. For example, the bioactive can also be present on those portions of the surface of the balloon between the cutting elements. In these embodiments, the bioactive can be present at a higher amount within the reservoir than on other portions of the surface. This embodiment can allow for the delivery of higher amounts of bioactive to the vessel wall at those regions contacted by the cutting elements.

In other embodiments, the cutting elements(s) do not include a reservoir. For example, the cutting elements can be raised or thickened portions of the balloon wall that do not have a depression at the apex. In these embodiments, a bioactive can be present on the surface of the balloon. For example, the bioactive may be coated onto all, or part, of the surface of the balloon. In such embodiments, the bioactive can be applied such that the amount of bioactive is uniform over the coated portion of the balloon. In other embodiments, the bioactive may be applied so as to achieve a higher amount of bioactive on the cutting elements and/or the regions of the balloon near to the cutting elements.

In one embodiment, a bioactive is applied directly to the surface of the reservoir. In another embodiment the bioactive is applied to a primer layer, which is placed directly on the surface of the reservoir. In certain embodiments, the bioactive is mixed with a carrier material and this mixture applied to the reservoir. As used herein, a “carrier material” refers to a material that forms a mixture with the bioactive.

In such a configuration, the release of the bioactive may be dependent on factors including the composition, structure and thickness of the carrier material. In one embodiment, the carrier material may contain pre-existing channels, through which the bioactive may diffuse, or channels created by the release of the bioactive, or another soluble substance, from the carrier material. In yet another embodiment, barrier layer is applied over the bioactive. The material of this barrier layer can be a porous material or biodegradable material. In certain embodiments, the material is a polymer.

The carrier material and/or the barrier layer can include, for example, a bioelastomer, PLGA, PLA, PEG, Zein, or a hydrogel. In some embodiments, the carrier material and/or the barrier layer includes microcrystalline cellulose, hydroxypropylmethyl cellulose, hydroxypropyl cellulose, a cellulose product, a cellulose derivative, a polysaccharide or a polysaccharide derivative. In other embodiments the carrier material and/or the barrier layer includes lactose, dextrose, mannitol, a derivative of lactose, dextrose, mannitol, starch or a starch derivative. In other embodiments, the carrier material and/or the barrier layer includes a biostable or a biodegradable material, for example, a biostable or a biodegradable polymer. Examples of such biostable and biodegradable polymers are disclosed in U.S. Publication Number 2004-0243225A1, published Dec. 2, 2004, the contents of which are incorporated by reference.

In certain embodiments, the primer layer includes, or is formed from, a material that causes the bioactive to be released from the implanted device in a shorter time than would be the case if the primer layer was not present. For example, if the bioactive material is a lipophilic material, such as paclitaxel, the primer layer can include a hydrophilic material, such as a hydrophilic polymer, in an amount that lessens the adhesion of the base material of the device to the lipophilic bioactive material and helps facilitate the release and delivery of the lipophilic material when the device is implanted. In other embodiments, the carrier material includes such a hydrophilic material.

The bioactive is applied to the reservoir and/or other portions of the balloon surface by spraying, dipping, pouring, pumping, brushing, wiping, vacuum deposition, vapor deposition, plasma deposition, electrostatic deposition, ultrasonic deposition, epitaxial growth, electrochemical deposition or any other method known to those skilled in the art. Where the bioactive is applied to only certain portions of the surface of the balloon, for example, the reservoirs of the cutting elements, other portions of the balloon may be masked so that they remain free of bioactive.

The bioactive can be, for example, a taxane, a taxane derivative, everolimus, zotarolimus, paclitaxel, rapamycin, a rapamycin derivative, an antisense oligonucleotide or a mTOR inhibitor. In other embodiments, the bioactive is a nitric oxide source such as sodium nitroprussiate, nitroglycerin, S-nitroso and N-nitroso compounds. In one embodiment, the bioactive is a material capable of releasing nitric oxide from blood-contacting surfaces. Examples of such materials include, but are not limited to, those described in U.S. publication number 2004/0224868A1, published Nov. 11, 2004, and 2002/0115559A1, published Aug. 22, 2002, the contents of which are incorporated by reference. Other examples of bioactive agents suitable for inclusion in the claimed devices are described in, U.S. Patent Publication Number 2010/0286594 published Nov. 11, 2010, the contents of which are incorporated by reference.

Another aspect of the present invention provides methods for treating a patient by breaking hardened plague on a vessel wall and dilating the vessel using the devices described herein. In some embodiments, the methods also include delivering a bioactive to the wall of the vessel. The term “treatment” or “treating” as used herein describes the management and care of a human or veterinary patient for the purpose of combating or preventing a disease, condition, or disorder and includes the administration of a bioactive to alleviate the symptoms or complications, or eliminate the disease, condition, or disorder.

The body vessel can include, but is not limited to, a vein, artery, biliary duct, ureteral vessel, body passage or portion of the alimentary canal. While many embodiments discussed herein discuss devices having application in the treatment of stenosis or restenosis, other embodiments provide for delivery to other body vessels.

In some embodiments, the present devices have a compressed delivery configuration with a very low profile, small collapsed diameter and great flexibility, and can navigate small or tortuous paths through a variety of body vessels. Such low-profile device can also be useful in coronary arteries, carotid arteries, vascular aneurysms, and peripheral arteries and veins (e.g., renal, iliac, femoral, popliteal, sublavian, aorta, intercranial, etc.) Other nonvascular applications include gastrointestinal, duodenum, biliary ducts, esophagus, urethra, reproductive tracts, trachea, and respiratory (e.g., bronchial) ducts.

The devices can be used to treat a narrowing of a peripheral artery or vein. Examples of such arteries include, but are not limited to, the femoral artery, the superficial femoral artery (artery below the branch for the profunda femoris artery), the popliteal artery and the infrapopliteal artery. Examples of such veins include, but are not limited to, the femoral vein, the popliteal vein and the lesser/greater saphenous vein.

The devices can be deployed according to well-known deployment techniques for expandable medical devices. For example, the device can be delivered to the point of treatment by advancing the catheter over a guidewire. The balloon is then expanded to bring the outside surface, and the cutting elements, into contact with the vessel wall. Further expansion of the balloon causes the cutting elements to breakup any hardened plague on the vessel wall, allowing further expansion of the vessel.

Expansion of the balloon can also result in a therapeutically-effective amount of a bioactive being delivered to the vessel wall. The bioactive may be delivered from the reservoir of the cutting element and/or from another surface of the balloon. In various embodiments, at least 90 percent of the bioactive present on the balloon catheter is released into an aqueous physiological environment within 30 sec, 1 minute, 2 minutes, 5 minutes, 10 minutes, 15 minutes, 20 minutes, 30 minutes, 45 minutes, 60 minutes or 90 minutes. After expansion of the vessel and delivery of the bioactive, the balloon is deflated and the catheter removed from the vessel.

In certain configurations, a rapid exchange delivery balloon catheter allows exchange from a balloon angioplasty catheter to a prosthesis delivery catheter without the need to replace the angioplasty catheter guide wire with an exchange-length wire guide before exchanging the catheters. Such delivery methods are described in U.S. Pat. Nos. 5,690,642, 5,814,061 and 6,371,961, the contents of which are incorporated by reference.

While preferred embodiments of the invention have been described, it should be understood that the invention is not so limited, and modifications may be made without departing from the invention. The scope of the invention is defined by the appended claims, and all devices that come within the meaning of the claims, either literally or by equivalence, are intended to be embraced therein. Furthermore, the advantages described above are not necessarily the only advantages of the invention, and it is not necessarily expected that all of the described advantages will be achieved with every embodiment of the invention.

I claim:

1. A device comprising:
a shaft having a distal end and a proximal end;
an inflatable balloon mounted at the distal end of the shaft, the shaft having a lumen extending therethrough and in fluid communication with an interior region of the balloon, and
a cutting element comprising a reservoir positioned on an exterior surface of the balloon, wherein the balloon comprises a wall of variable thickness, wherein the cutting element comprises a thickened portion of a wall of the balloon, and wherein the reservoir comprises a depression in the thickened portion.

2. The device of claim 1, further comprising a bioactive positioned within the reservoir.

3. The device of claim 2, wherein the bioactive is selected from the group consisting of a taxane, a taxane derivative, everolimus, zotarolimus, paclitaxel, rapamycin, a rapamycin derivative, an antisense oligonucleotide, and a mTOR inhibitor.

4. The device of claim 2, wherein the bioactive is paclitaxel.

5. The device of claim 2, further comprising a coating comprising the bioactive on a portion of the external surface of the balloon other than the reservoir.

6. The device of claim 1, comprising a plurality of cutting elements with reservoirs.

7. The device of claim 1, wherein the cutting element comprises a ridge extending across the external surface of the balloon.

8. The device of claim 7, wherein the reservoir comprises a depression formed in an apex of the ridge between two ridge walls, wherein the two ridge walls are configured to move apart upon inflation of the balloon.

9. The device of claim 1, wherein the wall of the balloon comprises a material selected from the group consisting of silicone, polyethyleneterepthalate (PET), polyvinyl chloride, polypropylene, polyethylene, polyurethanes, nylons, polyesters, latex, natural rubber, synthetic rubber, elastomers and mixtures or copolymers thereof.

10. The device of claim 1, wherein the wall of the balloon comprises a polyamide.

11. The device of claim 1, wherein the cutting element extends across the surface of the balloon in a helical or serpentine form.

12. The device of claim 1, wherein the balloon comprises a first end and a second end and wherein the cutting element extends a least part of the way from the first end to the second end.

13. The device of claim 1, wherein the cutting element and the balloon are molded as an integral unit.

14. A method for delivering a bioactive to a vessel wall, comprising
inserting a device into the vessel, wherein the device comprises
a shaft having a distal end and a proximal end;
an inflatable balloon mounted at the distal end of the shaft, the shaft having a lumen extending therethrough and in fluid communication with an interior region of the balloon, and
a cutting element positioned on an exterior surface of the balloon, wherein the cutting element comprises a reservoir containing the bioactive;
positioning the inflatable balloon within the vessel;
inflating the inflatable balloon, whereby the cutting element contacts the vessel wall;
maintaining the cutting element in contact with the vessel wall for a time period sufficient to deliver at least a portion of the bioactive to the vessel wall.

15. The method of claim 14, wherein the reservoir comprises a depression formed at an apex of the cutting element between two cutting element walls, wherein the two cutting element walls are configured to move apart upon inflation of the balloon.

16. The method of claim 14, wherein the bioactive is selected from the group consisting of a taxane, a taxane derivative, everolimus, zotarolimus, paclitaxel, rapamycin, a rapamycin derivative, an antisense oligonucleotide, and a mTOR inhibitor.

17. The method of claim 14, wherein the bioactive is paclitaxel.

18. A device comprising:
a shaft having a distal end and a proximal end;
an inflatable balloon mounted at the distal end of the shaft, the shaft having a lumen extending therethrough and in fluid communication with an interior region of the balloon, and
a cutting element positioned on an exterior surface of the balloon and extending across the surface of the balloon in a helical or serpentine form, wherein the cutting element comprises a thickened portion of a wall of the balloon and comprises a reservoir containing paclitaxel, and wherein the wall of the balloon comprises a polyamide.
